(19) United States
(12) Patent Application Publication
(10) Pub. No.: US 2023/0126362 A1
(43) Pub. Date: Apr. 27, 2023

(54) DRAIN-COMPATIBLE TISSUE EXPANSION DEVICE

(71) Applicant: Allergan, Inc., Irvine, CA (US)

(72) Inventors: David J. SCHUESSLER, Santa Ana, CA (US); Gina M. NUTI, Newport Beach, CA (US)

(21) Appl. No.: 17/920,775

(22) PCT Filed: Apr. 23, 2021
(86) PCT No.: PCT/US2021/028993

§ 371 Date: Oct. 21, 2022

Related U.S. Application Data
(60) Provisional application No. 63/015,403, filed on Apr. 24, 2020.

(51) Int. Cl. A61B 90/00; A61M 27/00

(52) U.S. Cl. CPC A61B 90/02; A61M 27/002; A61F 2250/0003; A61M 2210/1007

(57) ABSTRACT

A drain-compatible soft tissue expander can incorporate a drainage structure that allows fluid to be removed from the body of a living subject. The soft tissue expander can include an expandable shell and a drainage structure that can, for example, assist in mitigating or eliminating seromas. The drainage structure can be coupled to the shell at a plurality of locations and be removable therefrom without damaging the expandable shell.

### CROSS-REFERENCE TO RELATED APPLICATIONS

The present application claims the benefit of and priority to U.S. Provisional Application No. 63/015,403, filed Apr. 24, 2020, the entirety of which is incorporated herein by reference.

### BACKGROUND

### Field

The present disclosure relates to prosthetic implants and, more particularly, to breast implants and tissue expanders that incorporate fluid drainage systems.

### Description of the Related Art

Implantable prostheses are commonly used to replace or augment body tissue. In the case of breast cancer, a mastectomy is sometimes necessary to remove some or all of the mammary gland and surrounding tissue, which creates a void. This void may be filled with a fluid-filled implantable prosthesis. The implant serves to support surrounding tissue and to maintain the appearance of the body. The restoration of the normal appearance of the body has an extremely beneficial psychological effect on post-operative patients, alleviating much of the shock and depression that often follows extensive surgical procedures.

In breast reconstruction, prior to implantation of a long-term prosthesis, it is common practice to utilize a more temporary implant, for example, what is known as a “tissue expander” in order to gradually create the space necessary for the long-term prosthesis. For example, in some situations, such as a mastectomy, the chest tissues may be flat and tight, and an expander can serve to prepare the body for receiving a long-term prosthesis. Tissue expanders can also be used in other places in the body to expand healthy tissue to replace a nearby defect such as a burn or scar. Essentially, a tissue expander comprises an inflatable body, having an inflation valve connected thereto. The valve may be formed into the inflatable body itself or may be remotely located and connected to the inflatable body by means of an elongated conduit.

The inflatable body of the tissue expander is placed subcutaneously in the patient, at the location of where tissue is to be expanded. The inflation valve, whether on the implant or remote thereto, is also subcutaneously positioned or implanted, and is configured to allow gradual introduction of fluid, typically saline, into the inflation body, by injection with a syringe. After gradual inflation at pre-determined intervals, the skin and subcutaneous tissues overlying the expander are consequently caused to expand in response to the pressure exerted upon such tissues by the inflatable body as solution is gradually introduced therein.

During this process of breast reconstruction, certain risks may be present, such as potential infection and seroma formation. A seroma is believed to form as a result of tissue irritation that causes fluid to be exuded from tissue surrounding the implant. Although a seroma may dissipate or go away entirely on its own as the body absorbs the exuded fluid, a seroma can be painful and if large enough, may justify intervention by a surgeon to drain the seroma using a surgical technique. Thus, seroma removal often becomes an important consideration if the seroma continues to increase in size, puts pressure on the area of surgery, the skin, or an organ, becomes painful, or demonstrates signs of infection or inflammation. Because of this, drains are used in almost all breast reconstruction cases with tissue expanders due to the nature of the surgery and the significant increased rate of effusion of fluid from the surrounding tissues that cannot be resorbed by the lymphatic system.

In general, seromas can be drained by fine-needle aspiration. In this process, a surgeon uses a needled syringe to access and aspirate the seroma, removing fluid as necessary. However, if severe enough, a surgeon may fit a drainage tube into the area of concern so as to permit continued and steady drainage of the seroma. The drainage tube can be used to passively collect and drain fluid or as a component of a closed suction treatment, similar to the needled syringe. Nevertheless, such surgical intervention raises the risk of infection or other complications.

### SUMMARY

In accordance with some embodiments disclosed herein is the realization that effective treatment to minimize or address seroma formation may be most effective if a drainage system is incorporated into the tissue expander during the placement of the tissue expander itself. The drainage system can be integrated into or couplable to the tissue expander. The tissue expander can comprise one or more drainage structures that can accommodate a drain tube. Optionally, the drain tube can be removable from the drainage structure and separable from the tissue expander.

In some embodiments, a drain-compatible soft tissue expander can incorporate a drainage structure that allows fluid to be removed from the body of a living subject. The soft tissue expander can include an expandable shell and a drain tube that is coupled to the shell at a plurality of locations. The drain tube can be removable therefrom without damaging the expandable shell.

Optionally, the soft tissue expander can include a suture tab structure coupled to the expandable shell along a perimeter thereof. The drain tube can be coupled to suture tab structure at a plurality of locations and being removable therefrom without damaging the expandable shell.

Some embodiments can include a drainage structure having one or more loops configured to support a drain tube. Each loop can be configured as an aperture that extends through a rim component of the tissue expander. The rim component can comprise one or more tabs, an annular ring, or sections of an annular ring that extend at least partially around the circumference of the tissue expander. The rim component can comprise a generally planar or flat structure that extends coplanar with a posterior plane of the tissue expander.

Optionally, the rim component can comprise a tab ring that has a plurality of tabs or petals that extend radially therefrom about the full or partial circumference of the tab ring. The tabs or petals of the tab ring can each comprise an aperture through which a drain tube can be threaded.

In accordance with some embodiments, the apertures of the drainage structure can all have a single, common diameter for accommodating a given maximum diameter drain tube. However, optionally, the apertures of the drainage structure can have varying diameters to permit or accommodate a specific diameter drain tube. For example, the drainage structure can comprise a first set of apertures that has a first diameter and a second set of apertures that has a second diameter, wherein the first and second diameters are different from each other. The drainage structure can comprise two, three, four, or more sets of apertures that have different diameters. Further, the apertures of the drainage structure can comprise different shapes, other than circular.

Optionally, the drainage structure can comprise an alternating pattern of apertures that have different characteristics. For example, the drainage structure can have a pattern of first and second apertures in which the first and second apertures alternate with each other and have different diameters.

In some embodiments, the drainage structure can also be configured to comprise a flexible material that allows one or more portions of the drainage structure to be folded against the tissue expander. For example, a drainage structure having a series of tabs can be manipulated to hide or stow one or more of the tabs against the tissue expander. This can permit the surgeon to better visualize the placement an engagement of the drain tube with the drainage structure or to permit the drain tube better access to surrounding tissue and fluid.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 is a front plan view of a soft tissue expander having a drainage structure, according to some embodiments of the present disclosure.

FIG. 2 is a side view of the soft tissue expander of FIG. 1.

FIG. 3 is a perspective view of various drains that can be used in combination with the drainage structure of the soft tissue expander, according to some embodiments of the present disclosure.

FIG. 4 is a front plan view of another embodiment of a soft tissue expander having a drainage structure, according to some embodiments of the present disclosure.

FIG. 5 is a front plan view of a suture rim for a soft tissue expander, according to some embodiments of the present disclosure.

FIG. 6 is a front plan view of a soft tissue expander incorporating the suture rim shown in FIG. 4, according to some embodiments of the present disclosure.

FIGS. 7A and 7B illustrate side views of a soft tissue expander having the suture rim of FIG. 4, wherein tabs of the suture rim have been deflected from an initial position in FIG. 7A to a folded position in FIG. 7B, according to some embodiments of the present disclosure.

FIG. 8 is a front plan view of yet another embodiment of a soft tissue expander having a drainage structure, according to some embodiments of the present disclosure.

FIGS. 9 and 10 illustrate optional features that can be incorporated into individual tabs of the suture rim, according to some embodiments of the present disclosure.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

As described above, although tissue expanders can be used to stretch and facilitate generation of additional native tissue for skin and soft tissue defects and for breast reconstruction, certain challenges arise in their use. To address some of these challenges, some improvements have been made to allow a surgeon to more accurately position the expander relative to surrounding anatomy and to secure the expander at a specific, desired location. For example, as disclosed in Applicant's copending U.S. Patent Publication No. 2020/0085526, the entirety of which is incorporated herein by reference, some devices have been developed that permit can have a plurality of tabs that have one or more colors or unique attributes and/or an orientation indicator visible along a shell of the device for assisting a clinician and orienting the device during the implantation procedure.

The present disclosure addresses these and other challenges and provides tissue expanders that can be used to substantially improve post-operative maintenance of the tissue expander. Further, the present disclosure also provides tissue expanders that can be designed to include a selectable complete or partial perimeter strip or band of suture tab-like material is provided to create a circumferential border so that the surgeon can choose the precise location(s) where to suture down the device for his/her preference or the needs of the patient.

In some embodiments, the present disclosure provides tissue expanders that can have a drainage structure to facilitate drainage of fluid from the breast cavity and thereby mitigate the occurrence and effect of seromas in a patient. Thus, some embodiments of the tissue expanders disclosed herein can include a drainage structure that can be integrated with and/or separable from the tissue expander, e.g., the shell, the suture tabs, and/or other structures of the tissue expander itself. Rather than requiring an additional operation to withdraw fluid or to insert a drain subcutaneously after a seroma has formed, tissue expanders in accordance with embodiments of the present disclosure can incorporate a drainage structure that can immediately, upon implantation, provide a fluid drain for improving patient comfort and recovery, and when no longer required, be quickly and easily disengaged from the tissue expander and removed from the patient without damaging the expandable shell.

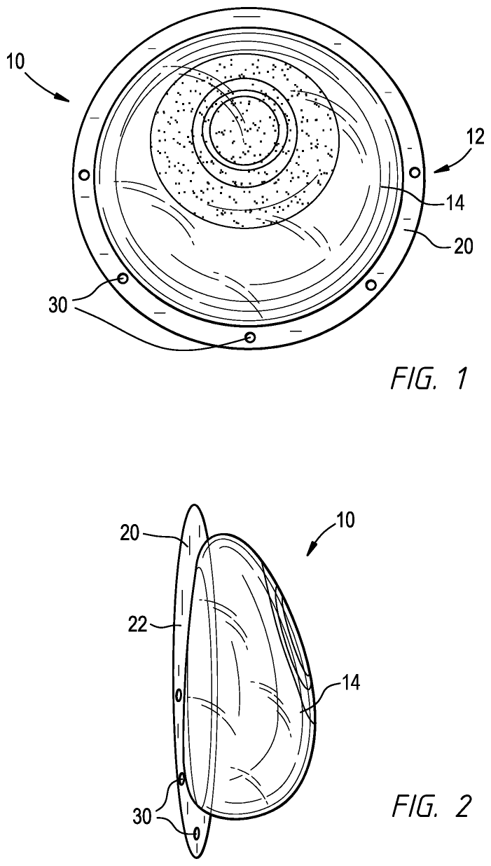

For example, referring to FIGS. 1 and 2, some embodiments disclosed herein relate to an improvement to a tissue expander 10 that comprises a drainage structure 12 coupled to a shell 14 of the tissue expander 10. The drainage structure 12 can comprise a suture strip or rim 20 that is coupled to a posterior surface 22 of the tissue expander 10. The rim 20 can comprise a single, continuous material that extends around a perimeter of the shell 14.

The drainage structure 12 can also include a drain component, such as a drain tube. The rim 20 can be designed to accommodate or support the drain tube. For example, in some embodiments, the rim 20 can comprise one or more apertures 30, which can be used to support a drain, as shown in FIGS. 1 and 2. The drain component can be woven or coupled to one or more apertures or other structures along the perimeter of the shell 14 to couple the drain component to a lower lobe or lower perimeter of the shell 14.

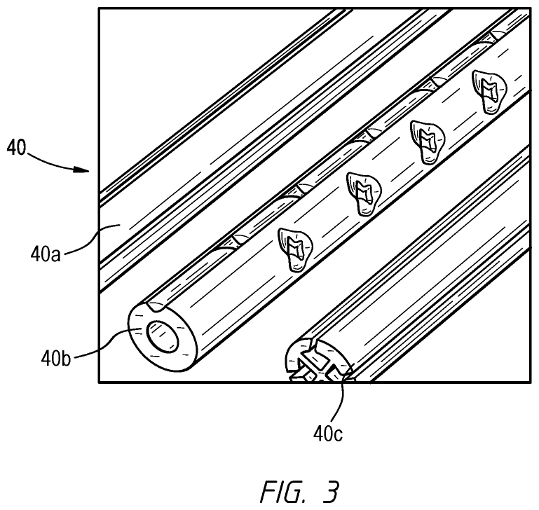

FIG. 3 illustrates several examples of drains or drain tubes 40 that can be used in combination with the tissue expander 10 shown in FIGS. 1 and 2, as well as other tissue expanders disclosed herein.

When assembled, a drain tube 40 can be placed, for example, in a fixed position, near a perimeter of the expander 10, such as through one or more of the apertures 30. For example, the drain tube 40 can be positioned in an inferior position near the inframammary fold, where excess fluid can collect.

Referring still to FIG. 3, the drain tube 40 can be configured as a flexible tubular member 40a having a central lumen. Further, in some embodiments, the drain tube 40 can be configured as a flexible tubular member 40b that comprises one or more side apertures, such as perforations that extend between an external surface of the drain and an inner lumen of the drain. The drain tube 40 can also be configured as a flexible tubular member 40c that comprises a series of lumens or channels that extend longitudinally along the length of the drain and that provide access to the lumens or channels via apertures or longitudinal slots that extend at least partially along the length of the drain, such as along an outer surface of the tubular member 40c. Such tubular members 40a, 40b, 40c can facilitate collection of fluid that is drained from the tissue and routing of the fluid to the drain exit, whether by natural, anatomical pressure or by suction.

In some embodiments, a proximal end of the drain tube 40 can exit the body through a separate puncture site in a lateral side of the chest. The proximal end of the drain tube 40 can be connected to a means of suction, for example a suction reservoir, that applies continuous negative pressure along the drain tube 40 to accumulate the fluid outside the body in the reservoir. Alternatively, the proximal end of the drain tube 40 could be provided with a self-sealing injection port end that could remain implanted in the lateral chest area.

Therefore, in some embodiments, fluid could then be regularly removed manually by percutaneous aspiration with a needle and applying negative pressure with a syringe or other means to remove any accumulated fluid. As noted above, if this fluid is not removed during the post-operative period, it can create swelling, produce a seroma and be a source of pain and infection. The drain tube 40 can be left in the tissue expansion site for one or two weeks until the healing processes reduces fluid creation to a rate that the lymphatic system can maintain. When fluid creation has subsided, the drain tube 40 is removed by pulling the external proximal end of the drain tube 40 from the original puncture wound leaving the tissue expander in place and without damaging the shell itself.

Optionally, some embodiments disclosed herein allow the drain tube 40 to be attached to the expander in the preferred locations desired by the surgeon and maintain the drain tube 40 in the desired location throughout the drainage process. The suture rim, for example, rim 20, can comprise a thin, flexible material. For example, suitable materials can include any suitable biocompatible polymers such as silicone, for example, polyester fabric reinforced silicone.

In addition, in some embodiments, the apertures or holes 30 may be disposed at regularly spaced intervals, for example, at regularly spaced apart about the perimeter of the tissue expander. The drainage structure can be configured such that the drain tube 40 can be inserted through one or more of the holes 30 to allow flexible, but exact attachment locations. The holes 30 can be sized slightly larger than a typical diameter of the drain tube 40 or be alternating sized holes to accommodate more than one size of drain tube. In some embodiments, the holes 30 may have diameters slightly smaller than a diameter of the drain tube 40, and are stretchable or expandable to allow receipt of the drain therethrough.

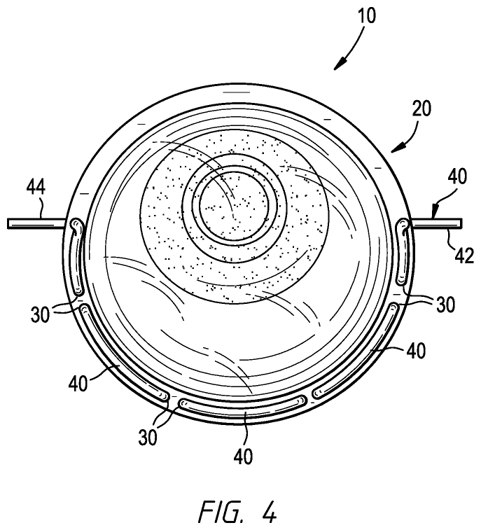

FIG. 4 illustrates a further embodiment of the tissue expander 10 in which the drainage structure 12 comprises both the suture rim 20 and a drain tube 40 that is threadingly engaged with the apertures 30 of the suture rim 20. As illustrated, the apertures 30 can advantageously maintain a position of the drain tube 40 in an inferior location along the perimeter of the tissue expander 10. Further, as also illustrated, the drain tube 40 can comprise a proximal end portion 42 and a distal end portion 44. The proximal end portion 42 can be configured to extend through a puncture wound to provide access to the drain tube 40, as noted above. The distal end portion 44 can be implanted within the patient's tissue and provide an open or closed end, which can facilitate further fluid collection from the seroma.

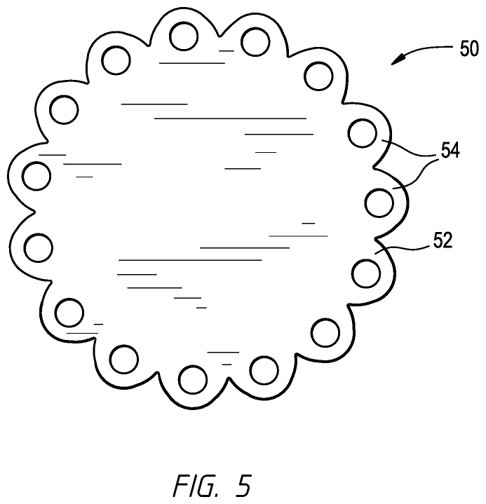

FIG. 5 illustrates an example drainage structure 50 that can comprise a suture rim 52 and a plurality of tabs or petals 54. In the illustrated embodiment, the suture rim 52 is in the configuration of a generally scalloped structure. The tabs 54 can extend in a radial direction and provide a variety of potential suture or drain connection locations on the drainage structure 50. The drainage structure 50 can be coupled to the posterior portion of a tissue expander, similar to that illustrated in FIGS. 1 and 2. In some embodiments, the suture rim 52 of the drainage structure 50 can comprise a high durometer silicone material, or a mesh reinforced silicone material, or a reinforced fabric or other material that allows the thickness of the suture rim 52 to be minimized, thereby improving flexibility of the suture rim 52. Alternatively, other materials, such as silicone, can be used in forming the drainage structure 50. The design and material choice of the suture rim 52 can enable a person of skill in the art to provide a thicker and stiffer structure or a thinner and more flexible structure, as well as to provide a desired tensile and shear strength, depending on the desired attributes and configuration of the suture rim 52.

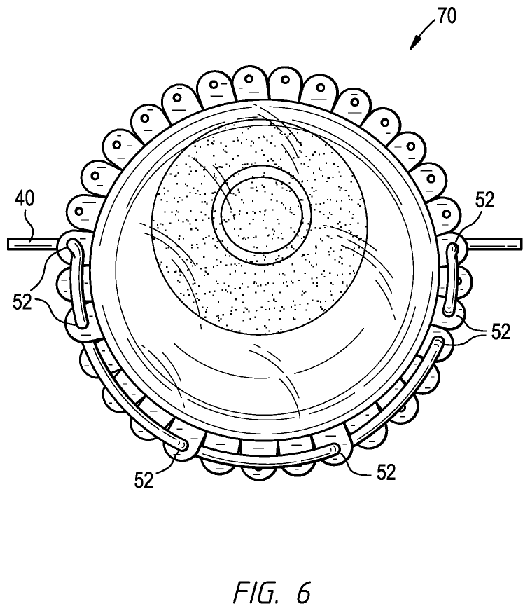

Thus, some embodiments can comprise a drainage structure 50 that is formed with a suture rim 52 with individual petals or tabs 54 that provide more flexibility in the attachment of the drain tube 40 which could make drain removal easier. An example of this design is shown in FIG. 6. Utilizing the drainage structure 50, an expander 70 can be formed, as shown in FIG. 6, by weaving a drain tube 40 into and through one or a few tab holes around the bottom half of the expander to keep the drain in place. The individual petals or tabs 54 of the suture rim 52 can be structured to provide a high degree of flexibility in the attachment of the drain tube 40, for example, in order to facilitate removal of the drain tube 40.

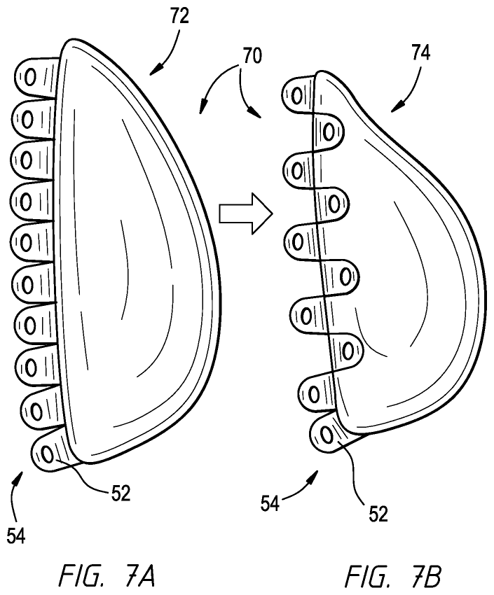

In accordance with some embodiments, each of the tabs 54 of the suture rim 52 can function or move independently of each other, e.g., by bending, rotating, or moving relative to an adjacent tab 54. For example, a given tab 54 may bend 180 degrees from its neighboring tab, thereby allowing only certain tabs 54 to be visible. Further, in some embodiments, certain tabs can be cut or removed from the suture rim 52 in order to allow the surgeon to create a specific tab design according to preferences and/or patient needs. Such adaptability is illustrated in FIGS. 7A and 7B, in which the tabs 54 of the suture rim 52 have been deflected from an initial position 72 in FIG. 7A to a folded position 74 in FIG. 7B. The tabs 54 of the suture rim 52 can be manipulated and bent so that one or more tabs, e.g., every other tab 54, can be deflected in an anterior direction relative to a neighboring tab 54, as shown in FIG. 7B. Thus, the tabs 54 of the tissue expander 70 can be manipulated between the first (initial) position 72 and the second (or folded) position 74.

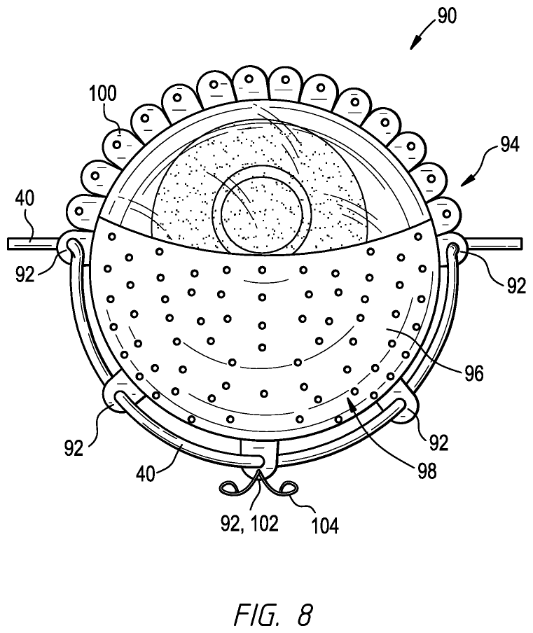

Optionally, in accordance with some embodiments, a wrap, such as an ADM (Acelluar Dermal Matrix), can be used with the expander. For example, FIG. 8 illustrates an embodiment of a tissue expander 90 in which individual tabs 92 of the drainage structure 94 can be utilized with a reinforcing tissue expander wrap or ADM 96, which may be used in breast reconstruction. Typically, the ADM 96 can be provided separately in sheet form and an inferior portion 98 of the expander 90 is wrapped with the ADM 96 by the surgeon on a back table, e.g. before implantation, and the assembly is then implanted.

In such an embodiment, individual tabs 92 of the suture rim 100 that the surgeon needs for drain attachment or suturing needs can be utilized by making appropriate slits in the ADM 96 during the wrapping process and pulling through those tabs 92 through such slits to extend outside the wrap. The remainder of the tabs (which not shown in FIG. 8) can be folded under the expander 90 and stowed inside the ADM 96 or can be cut away, as desired. The drain tube 40 can be attached as needed and thereafter, the expander 90 is ready for implantation.

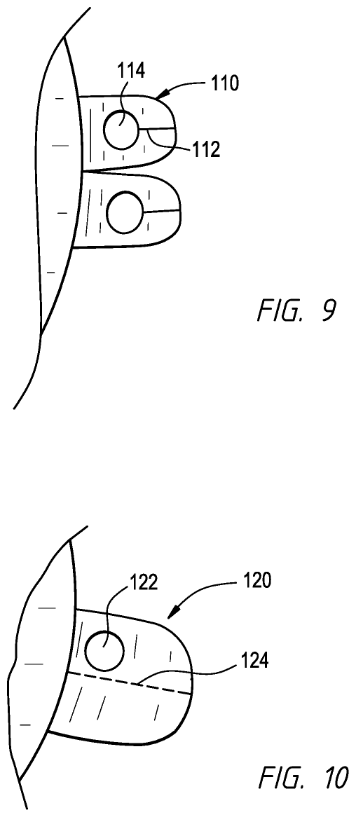

An additional feature that could be used to make the drain attachment easier, is to provide a narrow opening or a slit in the tab, extending from an outer edge to an end or side of each aperture, or a similar feature, in the individual tabs so that the end of the drain tube 40 does not need to be weaved through the various tab holes, but rather pushed through the slit to the aperture, or almost snapped in place. Such an embodiment is shown in FIG. 9.

The flexible single design of the embodiment of the tissue expander with the individual tabs could be provided to the surgeon and used in a typical breast reconstruction, being fully functional with or without a drain tube 40 and/or ADM 96.

In accordance with some embodiments, FIG. 9 illustrates that one or more of the tabs 110 of a drainage structure can be modified to include a slit or perforation 112, or a frangible section to permit preferential full or partial tearing of the tab during removal of the drain tube, which can facilitate maintaining the shell intact and without damage upon removal of the drain tube.

For example, after the expander has been implanted for a sufficient period of time and drainage is complete, the drain tube (which has been threaded into one or more apertures of the implanted expander) can be removed without damaging the shell. With the tensile, torsional, and/or shear stresses exerted on the tabs by the drain tube during removal, the slit 112 of the tab 110, for example, a frangible slit 112, can tear in order to increase the size of the aperture 114 or to fully tear through the tab, thereby releasing the drain tube from engagement with the tab.

In addition or alternatively to tabs that have tearable slits, one or more tabs of the drainage structure can be modified to include an open slit (i.e., one that is not perforated), thereby permitting a surgeon to assemble the extender with the drain tube by snapping it in place. For example, such an embodiment can be used to make the drain attachment easier, by providing an open slit (i.e., not perforated or tearable, but having separate edges) that leads from a perimeter of the tab to an edge of the aperture, or a similar feature, so that the end of the drain tube does not need to be weaved through the various tab holes, but rather almost snapped in place.

Optionally, as noted above with regard to some embodiments, the drain-diameter-to-hole-size for each aperture can be specifically tailored according to a pattern or a designed tolerance or fit. For example, it may be desirable to use a drain tube of a given size, such as between about 5 French and 15 French, such as about 7 French, 8 French, 9 French, or 10 French. In such embodiments, the size of the apertures can be between about 5% to about 50% larger than the size of the drain tube in order to accommodate a certain fit or configuration. For example, if perforated, tearable slits are present in the attachment tabs of the suture rim to which the drain tube is threadingly coupled, it may be beneficial to have a tighter tolerance or fit that allows ease for assembly, but permits the drain tube to be able to exert a substantial frictional force against the aperture and slit to induce tearing of the slit during removal. Further, if no slits are used, it may be desirable to have a larger difference between the outer diameter of the drain tube and the inside diameter of the apertures. Moreover, as noted above, any of the embodiments disclosed herein can incorporate multiple aperture sizes or patterns that allow a single expander to be used with a variety of different drain tube sizes a suitable drain tube size based on the patient's need.

Optionally, in some embodiments, as illustrated in FIG. 10, a drainage structure can be configured such that a size, longitudinal extent, shape, or width of the tab 120 is configured relative to or corresponds to a desired size, diameter, or shape of the aperture 122. For example, a width of the tab 120 may be one, two, three, or four times as great as a diameter of the aperture 122. FIG. 10 illustrates an embodiment in which the diameter of the aperture 122 is about one third of the width of the tab 120. Further, the aperture 122 is positioned off-center from a midline 124 of the tab, thereby allowing a surgeon to easily utilize a substantial portion of the tab 120 for suturing the tissue expander to the patient's anatomy. Various other advantages and benefits can be achieved by locating the aperture in an off-center location on the tab, not just structural advantages for anchoring via suture, but also reducing a slit length if a slit is used to extend from an edge of the tab to the aperture. Additionally, placing the aperture closer to the expander, rather than at a distal location on the tab, may allow the surgeon to more easily remove the drain tube because the aperture has a reduced or minimal lateral deflection relative to an edge of the expander and therefore provides a more predictable (minimal) movement when the drain tube is being pulled out of engagement with the drainage structure.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A soft tissue expander for implanting into a body of a living subject, the soft tissue expander comprising: an expandable shell having an anterior portion, a posterior portion, and a perimeter; and a drainage structure coupled to the posterior portion of the expandable shell, the drainage structure comprising an engagement member and a drain tube being removably couplable to the engagement member about at least a portion of the shell perimeter.

Clause 2. The soft tissue expander of Clause 1, wherein the engagement member comprises a plurality of tabs coupled to the posterior portion of the shell.

Clause 3. The soft tissue expander of Clause 2, wherein each of the plurality of tabs comprises an aperture to permit insertion of the drain tube therethrough.

Clause 4. The soft tissue expander of Clause 3, wherein each of the plurality of tabs comprises a frangible slit extending from a perimeter of the tab to the aperture of the tab to permit the tab to selectably tear along the slit to permit removal of the drain tube therefrom.

Clause 5. The soft tissue expander of any one of the preceding Clauses, wherein the engagement member comprises a scalloped tab ring coupled to the posterior portion of the shell.

Clause 6. The soft tissue expander of Clause 5, wherein the scalloped ring comprises a plurality of tabs, each of the plurality of tabs comprising an aperture to permit insertion of the train tube therethrough.

Clause 7. The soft tissue expander of any one of the preceding Clauses, wherein the engagement member extends coupled to the posterior portion of the shell.

Clause 8. The soft tissue expander of any one of the preceding Clauses, wherein the drain tube comprises a size of at least 5 French.

Clause 9. The soft tissue expander of any one of the preceding Clauses, wherein the drain tube comprises a size of between 5 French and 15 French.

Clause 10. The soft tissue expander of any one of the preceding Clauses, wherein the drain tube comprises a size of between 7 French and 10 French.

Clause 11. The soft tissue expander of any one of the preceding Clauses, wherein the drainage structure comprises a plurality of engagement apertures for permitting slidable engagement with the drain tube.

Clause 12. The soft tissue expander of Clause 11, wherein a size of the apertures is between about 5% to about 50% greater than a size of the drain tube.

Clause 13. The soft tissue expander of Clause 11, wherein the drainage structure comprises a first set of apertures and a second set of apertures, wherein a size of the first set of apertures is different from a size of the second set of apertures.

Clause 14. The soft tissue expander of Clause 13, wherein the drainage structure is configured such that apertures of the first set of apertures alternate with apertures of the second set of apertures.

Clause 15. The soft tissue expander of Clause 11, wherein the drainage structure comprises three sets of differently sized apertures, the three sets of differently sized apertures being positioned in a pattern about a perimeter of the drainage structure.

Clause 16. The soft tissue expander of any one of the preceding Clauses, wherein the drainage structure extends about only an inferior portion of the expander.

Clause 17. The soft tissue expander of any one of the preceding Clauses, further comprising an acelluar dermal matrix.

Clause 18. A soft tissue expander for implanting into a body of a living subject, the soft tissue expander comprising: an expandable shell having an anterior portion, a posterior portion, and a perimeter; and a drainage structure coupled to the posterior portion of the expandable shell, the drainage structure comprising an engagement member for engaging with a drain tube for removable coupling of the drain tube to the engagement member about at least a portion of the shell perimeter.

Clause 19. The soft tissue expander of any one of Clause 18, further comprising a drain tube.

Clause 20. The soft tissue expander of any one of Clauses 18 to 19, further comprising an acelluar dermal matrix.

Clause 21. A soft tissue expander for implanting into a body of a living subject, the soft tissue expander comprising an expandable shell and a drainage structure coupled to the expandable shell and extending along the perimeter thereof.

Clause 22. The soft tissue expander of any one of Clause 21, wherein the drainage structure comprises an engagement member and a drain tube that is removably couplable to the engagement member about at least a portion of the shell perimeter.

Clause 23. The soft tissue expander of any one of Clauses 21 or 22, wherein the drainage structure is coupled adjacent to a posterior portion of the expandable shell.

Clause 24. The soft tissue expander of any one of Clauses 21-23, wherein the drainage structure comprises a drain tube woven into a tab structure, the tab structure being coupled to the shell.

Clause 25. The soft tissue expander of any one of Clauses 21-24, wherein the drainage structure extends about a lower portion of the shell.

Clause 26. The soft tissue expander of any one of Clauses 21-25, wherein the drainage structure comprises a drain tube having a plurality of apertures.

Clause 27. The soft tissue expander of any one of Clauses 21-26, wherein the drainage structure comprises a drain tube having a plurality of longitudinal slots.

Clause 28. A soft tissue expander for implanting into a body of a living subject, the soft tissue expander comprising an expandable shell, a suture tab structure coupled to the expandable shell along a perimeter thereof, and a drain tube coupled to suture tab structure at a plurality of locations and being removable therefrom without damaging the expandable shell.

Clause 29. The soft tissue expander of Clause 28, wherein the suture tab structure comprises a plurality of extendible tabs.

Clause 30. The soft tissue expander of any one of Clauses 28 or 29, wherein each of the plurality of extendible tabs is moveable relative to the perimeter of the expandable shell between a retracted position and an extended position.

Clause 31. The soft tissue expander of any one of Clauses 28-30, wherein the drain tube is woven into the suture tab structure.

Clause 32. The soft tissue expander of any one of Clauses 28-31, wherein the drain tube comprises a plurality of apertures.

Clause 33. The soft tissue expander of any one of Clauses 28-32, wherein the drain tube comprises a plurality of longitudinal slots.

Clause 34. The soft tissue expander of any one of Clauses 28-33, further comprising an acelluar dermal matrix.

### Further Considerations

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

1. A soft tissue expander for implanting into a body of a living subject, the soft tissue expander comprising:
an expandable shell having an anterior portion, a posterior portion, and a perimeter; and
a drainage structure coupled to the expandable shell and extending along the perimeter thereof, the drainage structure comprising an engagement member and a drain tube being removably couplable to the engagement member about at least a portion of the shell perimeter.

2. The soft tissue expander of claim 1, wherein the engagement member comprises a plurality of tabs coupled to the posterior portion of the shell.

3. The soft tissue expander of claim 2, wherein each of the plurality of tabs comprises an aperture to permit insertion of the drain tube therethrough.

4. The soft tissue expander of claim 3, wherein each of the plurality of tabs comprises a frangible slit extending from a perimeter of the tab to the aperture of the tab to permit the tab to selectably tear along the slit to permit removal of the drain tube therefrom.

5. The soft tissue expander of claim 1, wherein the engagement member comprises a scalloped tab ring coupled to the posterior portion of the shell.

6. The soft tissue expander of claim 5, wherein the scalloped ring comprises a plurality of tabs, each of the plurality of tabs comprising an aperture to permit insertion of the train tube therethrough.

7. The soft tissue expander of claim 1, wherein the engagement member extends coupled to the posterior portion of the shell.

8. The soft tissue expander of claim 1, wherein the drainage structure comprises a plurality of engagement apertures for permitting slidable engagement with the drain tube.

9. The soft tissue expander of claim 8, wherein a size of the apertures is between about 5% to about 50% greater than a size of the drain tube.

10. The soft tissue expander of claim 8, wherein the drainage structure comprises a first set of apertures and a second set of apertures, wherein a size of the first set of apertures is different from a size of the second set of apertures.

11. The soft tissue expander of claim 10, wherein the drainage structure is configured such that apertures of the first set of apertures alternate with apertures of the second set of apertures.

12. The soft tissue expander of claim 8, wherein the drainage structure comprises three sets of differently sized apertures, the three sets of differently sized apertures being positioned in a pattern about a perimeter of the drainage structure.

13. The soft tissue expander of claim 1, wherein the drainage structure extends about only an inferior portion of the expander.

14. A soft tissue expander for implanting into a body of a living subject, the soft tissue expander comprising an expandable shell, a suture tab structure coupled to the expandable shell along a perimeter thereof, and a drain tube coupled to suture tab structure at a plurality of locations and being removable therefrom without damaging the expandable shell.

15. The soft tissue expander of claim 14, wherein the suture tab structure comprises a plurality of extendible tabs.

16. The soft tissue expander of claim 14, wherein each of the plurality of extendible tabs is moveable relative to the perimeter of the expandable shell between a retracted position and an extended position.

17. The soft tissue expander of claim 14, wherein the drain tube is woven into the suture tab structure.

18. The soft tissue expander of claim 14, wherein the drain tube comprises a plurality of apertures.

19. The soft tissue expander of claim 14, wherein the drain tube comprises a plurality of longitudinal slots.

20. The soft tissue expander of claim 14, further comprising an acelluar dermal matrix.
